(19) United States
(12) Patent Application Publication
(10) Pub. No.: US 2024/0002787 A1
(43) Pub. Date: Jan. 4, 2024

(54) POROUS GLASS-BASED MICROBIAL STORAGE AND DELIVERY SYSTEM

(71) Applicant: BATTELLE SAVANNAH RIVER ALLIANCE, LLC, AIKEN, SC (US)

(72) Inventors: ROBIN L. BRIGMON, NORTH AUGUSTA, SC (US); ALEX J. KUGLER, AIKEN, SC (US); CORY L. TRIVELPIECE, AIKEN, SC (US)

(21) Appl. No.: 17/855,141

(22) Filed: Jun. 30, 2022

(51) Int. Cl. C12N 5/00; B09C 1/10; C02F 3/32; C02F 3/34

(52) U.S. Cl. CPC C12N 5/0068; B09C 1/10; B09C 1/105; C02F 3/322; C02F 3/347; C02F 2101/32; C12N 2533/14; C12N 2533/12; C12N 2535/00; B09C 2101/00; C02F 2305/06; C02F 3/343

(57) ABSTRACT

Solid, porous, glass-based substrate for storage and delivery of useful microorganisms are described. Materials include one or more microorganisms lyophilized on the substrate, e.g., in the form of a lyophilized biofilm. The materials can be utilized for long-term storage, transport, and deployment of one or more microorganisms, such as consortium of microorganisms at a remediation site.

### FEDERAL RESEARCH STATEMENT

This invention was made with Government support under Contract No. 893033210EM00080, awarded by the U.S. Department of Energy. The Government has certain rights in the invention.

### BACKGROUND

Addition of living microorganisms, e.g., bacterial amendments, are beneficially used in a plurality of applications, including oil degradation, mining, bioremediation of contaminated waters and soils, agriculture, water treatment, and even within the human body. However, the addition of microorganisms to a system can also create unintended consequences. For instance, effective wastewater treatment is of paramount importance due to the impact of municipal, industrial, and medical wastewater on natural water, soil, and agricultural products. Effective wastewater treatment is of particular concern in highly populated areas. Wastewater treatment plants make use of beneficial bio-based reactions with activated sludges, aerated lagoons, ponds, and wetlands, among other techniques. However, overuse or inadvertent release of microorganism amendments used in wastewater treatment can lead to ecological damage, as well as health dangers to people and animals, affected by the misuse or release.

Cell cultures are a common part of bioremediation strategies, but they can be difficult to deploy. Aerobic bacteria in particular can be extremely difficult to deploy in the field. Cultures can die in transport or fail to proliferate and must be chosen specifically for each deployment environment. Liquid bioremediation cultures are commonly developed and, while they can be deployed in the field, the process is difficult, expensive, and requires considerable regulatory oversight, making it desirable to find alternatives means of deployment.

Bioremediation strategies have also looked to the development and use of biofilms. Biofilms are used in water filtration systems, wastewater remediation, biocatalysts, and the generation of biofuels. Such films often have high cell densities, e.g., 10⁸-10¹¹ cells per gram of wet biomass. While biofilms can form from a single species, they often consist of many different species living together. This diversity aids in their survival and enhances their biotechnological use by allowing coordination of life cycles by staggering the expression of certain genes or proteins. Biofilms also have enhanced gene exchange due to cell/cell proximity and high cell density which increases the resiliency of the bacteria compared to their free-living counterparts. Considerable research has focused on biofilm growth on various surfaces such as rocks, well screens, and ships. Unfortunately, however, these surfaces are of limited biotechnical application and many desirable microbes have been difficult or impossible to develop in the form of biofilms on such surfaces.

Needed in the art are methods and systems for deployment of microorganism amendments. Methods and systems that can be utilized to store and deploy beneficial microorganisms where necessary efficiently and with relative ease, for instance, in the form of a solid supported biofilm, would be of great benefit to the art.

### SUMMARY

According to one embodiment, disclosed is a microorganism storage and delivery system. A system can include a solid, porous substrate that includes a glass-based material, e.g., a natural or synthetic foamed glass or a foamed glass ceramic (FGC). A system can also include a lyophilized microorganism (e.g., bacterial, fungal, algal, etc.) on the surface of the solid, porous substrate. For instance, the microorganism can be in the form of a lyophilized biofilm on the surface of the solid, porous substrate.

Also disclosed are methods for processing a microorganism for delivery to a site of use, e.g., a bioremediation site. A method can include culturing the microorganism(s) in the presence of a solid, porous substrate that includes a glass-based material. Following culturing, the microorganism(s) can be lyophilized on the surface of the substrate. Thereafter (e.g., following storage and transport), the composite material including the lyophilized microorganism on the surface of the substrate can be delivered to a site of use and placed in a suitable culture environment, where the microorganism can again grow and develop. Upon regrowth, the microorganism can provide beneficial use at the site. Optionally, the composite material can be collected following a period of time during which the microorganism carries out the beneficial use, e.g., degradation of contaminants in the environment.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a porous solid support as encompassed herein.

FIG. 2 schematically illustrates a microbial delivery system as disclosed herein.

FIG. 3A illustrates a diffractogram of an FGC utilized as a substrate in the example section herein and showing the various mineral phases in the FGCs.

FIG. 3B illustrates a scanning electron microscope (SEM) image of the FGC of FIG. 4A.

FIG. 3C illustrates an SEM of the FGC of FIG. 4A at higher magnification.

FIG. 3D illustrates an SEM of the FGC of FIG. 4A at higher magnification.

FIG. 4 illustrates a growth curve of a representative bacterial consortium as a function of time.

FIG. 5 illustrates biofilms after one week of incubation in the presence of FGCs, persevered with glycerin and then lyophilized. Algal samples (A and B), BioTiger™ consortium (C and D) and Bacillus thuringiensis (E and F) show clearly defined and lyophilized extracellular polymeric substance which has the microorganisms incorporated within.

FIG. 6 illustrates growth curves of a bacterial sample from a preserved state on FGCs.

FIG. 7 presents total hydrocarbon remaining in test soil following various treatments including materials as described herein.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to the utilization of a solid, porous, glass-based substrate for storage and delivery of useful microorganisms. More specifically, disclosed are composite materials that include a solid, porous, glass-based substrate and one or more microorganisms lyophilized on the substrate, e.g., in the form of a lyophilized biofilm.

The materials can be utilized for long-term storage, transport, and delivery of one or more microorganisms in a variety of useful applications. For instance, and without limitation, the biologically amended composite materials can be formed to include single or a consortium of microorganisms for deployment to mitigate disasters (e.g., oil spills), remediate legacy contamination (e.g., acid mine drainage), utility applications (e.g., wastewater treatment), agricultural applications (e.g., soil restoration), etc. Moreover, due to the nature of the solid, porous substrate of the materials, the materials can be easily recovered following use, preventing the release of the microorganisms retained thereon, as well as preventing overuse of the microorganisms at a deployment site.

Solid substrates of the composite materials are environmentally friendly, inexpensive, and capable of supporting the microorganism(s) of choice. The solid substrates can be porous and glass-based. As such, the lyophilization process can be easier as compared to previously known materials, e.g., unsupported microorganism cultures and substrates not conducive to microorganism attachment, growth, and development. Moreover, the lyophilized microorganism retained on the glass-based materials need only be placed in a receptive environment for growth and development to again begin, whereas previously known lyophilized cultures often require a lengthy growth period in a laboratory environment following dormancy. The solid glass-based materials can also provide for long shelf life and easy transport without the need for expensive environmental control, as well as facile reclamation of the materials, following use.

The glass-based, porous supporting substrate can be a natural or synthetic material. In one embodiment, a glass-based, porous supporting substrate can include post-consumer recycled glass. For instance, a substrate can include a synthetic foamed glass that includes a recycled glass. Moreover, in some embodiments, the nature of the solid supporting substrate material allows for further reuse. For instance, the following deployment and recovery, a substrate material can be autoclaved and reused making them recyclable and further decreasing costs.

As utilized herein, the term “glass” generally refers to a non-crystalline silicate material. In some embodiments, a glass of a porous, solid substrate can include silicon dioxide. However, a glass of a solid substrate is not limited to any particular silicate material and can encompass, without limitation, natural pumice or synthetic equivalents, natural obsidian or synthetic equivalents, natural perlite or synthetic equivalents, soda-lime glass, flint, container glass, a-glass, flat glass, e-glass, c-glass, ar-glass, s-glass, niobophosphate glass, single-phase borosilicate glass, phase separated borosilicate glass, fused silica, coal slags, metal slags, smelting slags, mineral wool, or ash byproducts from incineration processes, as well as any combinations thereof.

A glass-based substrate can include a glass in conjunction with one or more additional materials, e.g., an amorphous material in conjunction with a secondary material, which can be a crystalline or non-crystalline material, including one or more different glasses. By way of example, and without limitation, a glass-based substrate can include one or more of alumina, alumina hydrate, aplite, feldspar, nepheline syenite, calumite, kyanite, kaolin, cryolite, antimony oxide, arsenious oxide, barium carbonate, barium oxide, barium sulfate, boric acid, borax, anhydrous borax, quicklime, calcium hydrate, calcium carbonate, dolomitic lime, dolomite, finishing lime, litharge, minium, calcium phosphate, bone ash, iron oxide, caustic potash, saltpeter, potassium carbonate, hydrated potassium carbonate, sand, diatomite, soda ash, sodium nitrate, sodium sulphate, sodium silica-fluoride, pyrolysis ash, zinc oxide, or any combination thereof.

In one embodiment, the glass-based material of the solid, porous substrate can include FGC, which is commonly described as a synthetic, pumice-like material. FGCs are porous glass and ceramic materials that can be formed almost entirely from waste glass or waste incinerator ashes. An FGC can have increased surface area compared to typical soda-lime glass and can be produced at temperatures significantly lower than the liquidus temperature of the original recycled glass cullet, as well as of a broad range of species, reactivity, and porosity characteristics. Beneficially, FGC can be produced with less environmental impact than traditional recycled products that require a full “re-melt” of the original glass.

The composition of an FGC can be controlled during pre-firing batching, and the physical properties of the materials can be controlled via chemical composition of the batch in addition to process parameters as described further in U.S. Patent Application Publications 2022/0073416, 2022/0081349, and 2022/0089476 all to Hust et al., which are incorporated herein by reference in their entirety. In some embodiments, an FGC can be formed to include one or more reactive agents that can interact with one or more substances when those substances contact the reactive agents, such as cementitious materials, pozzolanic materials, activated carbon, and/or clayey or zeolitic minerals.

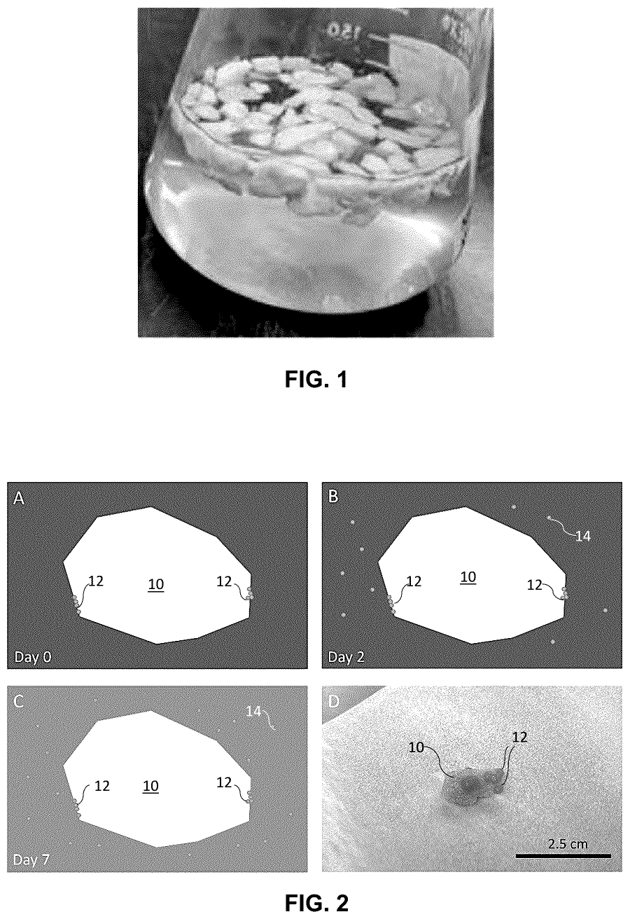

FIG. 1 presents an image of composite material including an FGC as a solid substrate as disclosed herein. As illustrated, the materials can be designed to be buoyant in a liquid system. Alternatively, through modification of chemical composition, the substrate materials can be designed for non-buoyancy, e.g., to fall to the bottom of a liquid deployment area, to be held between immiscible liquid phases, to disperse throughout a liquid, etc.

As indicated, the solid support media may include open-celled porosity, i.e., individual pieces of the media may include passageways extending from an external surface of the piece to the interior and/or to a second external surface of the piece. The porosity can provide high surface area to the substrate for supporting a high density of microorganism to be developed thereon. The size of the pores can be pre-determined to encourage growth and development of a microorganism of interest. For instance, when considering utilization of the materials for use with a relatively small microorganism, e.g., a bacterial organism or a small algae, the porosity can be relatively small, with average pore size on the order of hundreds of micrometers, e.g., from about 50 micrometers to about 1000 micrometers, or from about 100 micrometers to about 500 micrometers in some embodiments, which can be a desirable pore size when considering utilization of microorganisms have a size of a few micrometers, e.g., about 1 micrometer to about 5 micrometers in size. When considering larger organisms, the materials can be selected/designed to include a larger porosity, e.g., from about 500 micrometers to about 2 millimeters, or even larger, in some embodiments.

In some embodiments, a substrate media can have a single, well-defined porosity. For instance, a silicate aggregate having a single composition with highly homogenous and/or uniform properties, e.g., a single density and a single porosity. In other embodiments, a more complex material can be utilized. For instance, an FGC can include vitreous materials contained at least partially within pores of the base substrate material, leading to regions of the magmatics that are mesoporous (less than about 100 micrometers in cross-section) and/or microporous (less than about 1 micrometer in cross-section).

The bulk density and surface area (e.g., BET surface area) are not particularly limited. For instance, in some embodiments, the solid substrate can have a bulk density of from about 0.1 grams per centimeter (g/cc) to about 2 g/cc, such as from about 0.8 to about 4 g/cc, or from about 1 g/cc to about 3 g/cc, in some embodiments. In general, BET surface area can be about 100 square meters per gram (m²/g) or less, such as about 5 m²/g or less, or about 2 m²/g or less, in some embodiments, e.g., from about 0.1 m²/g to about 1 m²/g.

The solid substrate can have any desirable shape and size. In general, the solid substrate can be in the form of an aggregate, i.e., a plurality of individual pieces, typically having a largest cross-sectional size on the order of about 50 millimeters or less, such as about 25 millimeters or less, such as from about 1 millimeter to about 20 millimeters, or from about 2 millimeters to about 10 millimeters, in some embodiments. The solid substrate can likewise be provided in any suitable bulk shape, e.g., spherical, polyhedral, or nebulous shapes, as well as a mixture of random bulk shapes, such as is illustrated in FIG. 1.

To form the composite materials, the glass-based, porous media are loaded with one or more microorganisms of choice. There is no particular requirement on the types, kinds, or number of microorganisms that can be loaded on the substrate materials, and through use of disclosed methods, different lines of bacterial, algal, and fungal cells can be preserved, stored, and regrown after storage for a period of from a few days to several months.

In one embodiment, a microbial consortium can be loaded on a substrate. By way of example, in one embodiment, a substrate material can be inoculated with BioTiger™ (described in U.S. Pat. No. 7,472,747, which is incorporated by reference herein for all purposes), which is a microbial consortia developed by extensive microbiology screening and characterization of samples collected from a waste lagoon. BioTiger™ includes isolates as indicated in Table 1, below, and has been shown to be feasible for degradation of in situ oils, as well as to increase hydrocarbon recovery from oil sands. Several of the bacteria in this strain have also been shown to produce biosurfactants, which can increase the efficiency of the bioremediation process by dispersing the hydrocarbons and causing them to be more easily degraded.

TABLE 1

| Isolate | Identification |
|---|---|
| CZOR-L1B (KN-1) | ALCALIGENES-PIECHAUDII SRS |
| BP-20 (KN-2) | RALSTONIA PICKETTII SRS |
| CZOR-L1Bsm | PSEUDOMONAS-PUTIDA BIOTYPE B SRS |
| (KN-3) | |
| BPB | FLEXIBACTER CF. SANCTI SRS |
| BPC | PSEUDOMONAS FREDRIKSBERGENSIS SRS |
| BPE | STAPHYLOCOCCUS WARNERI. LMG 19417 SRS |
| BPF | SPHINGOMONAS SRS |
| BPH | SPHINGOMONAS SP. S37 SRS |
| BPI | PHYLOBACTERIUM SRS |
| | (α PROTEOBACTERIUM TA-A1) |
| BPJ | SERRATIA FICARIA SRS |
| | (α PROTEOBACTERIUM TA12-21) |
| BPK | AGROBACTERIUM TUMEFACIENS SRS |
| BPL | RHIZOBIUM SP. SDW045 SRS |

Other microorganisms as may be carried by use of a substrate as described can include, without limitation, Bacillus thuringiensis, Bacillus subtilis, Bacillus cereus, Bradyrhizobium spp., Thiobacillus ferrooxidans, Azospirillum spp., Azotobacter spp., Pseudomonas Putida, Pseudomonas fluorescens, Enterobacter spp., Penicillium spp., Aspergillus spp., Gluconobacter oxydans, Gluconacetobacter xylinus, Nitrosomonas spp., Nitrosococcus spp., Clostridium spp., Acidovorax delafieldii, Rhodococcus spp., Alcanivorax borkumensis, Halomonas spp., Vibrio gazogenes, Marinobacter hydrocarbonoclasticus, and Sphingomonas paucimobilis, just to name a few. Moreover, as with the BioTiger™ consortium mentioned herein, while individual microorganisms can be utilized alone, in some embodiments, a mixture of microorganisms can be utilized, and the resulting product can exhibit enhanced effect. For instance, one or more oil-degrading bacteria as are known in the art can be loaded on a porous substrate, optionally in conjunction with additional microorganisms, and the resulting product can exhibit a synergetic effect.

The method utilized for immobilization of the microorganism(s) on the substrate is not particularly limited and generally includes culturing the microorganisms in a suitable culture environment in the presence of the porous substrate materials. In some embodiments, an immobilization process can be similar to that of natural systems in the formation of biofilms. Beneficially, through utilization of disclosed materials and methods, microorganisms that do not preferentially live in a biofilm form can be immobilized on a solid porous glass-based substrate as described as a biofilm.

Biofilms are ubiquitous throughout the environment, both natural and synthetic. A biofilm is a consortium of sessile microorganisms that have established a three-dimensional community including of a combination of prokaryotic or eukaryotic cells embedded in a microbially produced matrix of extracellular polymeric substances (EPS). The EPS can include proteins, polysaccharides, humic substances, extracellular DNA, as well as additional molecules. Social and physical interactions occur intercellularly in conjunction with the EPS creating a unique and emergent lifestyle, distinctly different from that of a free-living microorganism. The unique properties of biofilms in comparison to their free-living cellular counterparts are well documented and can include increased antibiotic resilience, increased mutations, consortia building, and increased quorum-sensing-regulated mechanisms. Thus, providing a microorganism amendment in the form of a solid supported biofilm can have an advantage in bioremediation applications as it can provide the microorganisms in a high surface area and high density format that can increase the interaction with environmental contaminants and deployment capacity among other benefits, in addition to providing the ability to incorporate beneficial nutrients for the microorganisms into/onto the solid substrate in conjunction with the microorganisms.

The solid support of disclosed composites can also provide a route to modification of bulk characteristics of the amendments during use. For instance, FIG. 1 illustrates a biofilm-coated porous, glass-based solid substrate floating in an aqueous media. By modifying the local environment of the composite, one or more characteristics, e.g., the buoyancy characteristics, of the composite material can be altered. For instance, by addition of iron to the system, the iron can bind the biofilm, and the composite material can sink as the biofilm binds the iron. Such an approach can be useful in modification of operational parameters during use of a composite material.

Prior to lyophilizing, nutrients (nitrogen (N), phosphorous (P), carbon (C), or other key minerals) can be added to the materials for specific applications as an amendment. For example, in an oil-contaminated environment with high carbon content, a preparation with added N and P could be advantageous. Such amendments can be easily added on or with the composite material and can be application (e.g., site) specific.

Following inoculation, the supported microorganisms can be lyophilized. In general, the lyophilization process can encompass a standard lyophilization process, e.g., with treatment of 10% glycerin, flash-freezing below the eutectic point of the sample (e.g., at −80° C.), and freeze drying. However, there are microorganisms that may benefit from a more modified process, e.g., a rapid lyophilization method or preservation using dimethyl sulfoxide, as is generally known in the art.

The composite materials including the lyophilized microorganisms thereon can be stored as desired, e.g., up to several months or more. Beneficially, the storage environment does not require any specialized environmental conditions. For instance, the materials can be stored in air at standard temperature and pressure conditions.

The composite material can likewise be shipped and deployed without the need for any specialized environmental conditions. For instance, the materials need not be subjected to laboratory processing to revitalize the preserved microorganisms. In many embodiments, the composite materials can simply be located in the deployment area, following which the retained microorganisms can begin to grow and develop while retained on the substrate.

FIG. 2 illustrates one embodiment of a system of microbial delivery provided by disclosed materials. Specifically, FIG. 2 at A illustrates a solid substrate 10 including areas 12 of biofilm adhered thereto that include the microorganism(s) of interest. At day 0 (A), the material is illustrated following deployment in an environment of interest, e.g., an oil contaminated soil. When placed in the environment, e.g., soil or water, in combination with nutrients for the microorganism (e.g., nitrogen, phosphorous, etc.), which can be naturally present in the environment, deployed in conjunction with the materials, and/or incorporated in/on the substrate 10 in conjunction with the microorganisms, the lyophilized microorganisms of the biofilm 12 can begin to grow and develop. For instance, FIG. 2 at B illustrates the beginning of proliferation of cells 14 of the biofilm 12 within the environment at day 2 following deployment, and C shows continued proliferation of cells 14 in conjunction with desired effect of the microorganisms in the environment at day 7 following deployment, e.g., decrease in total hydrocarbon concentration in the soil as indicated by change in background shade of the image. At D of FIG. 2 is illustrated an exemplary composite material, which includes an FGC substrate particle 10 with several live BioTiger™ biofilms 12 thereon.

Disclosed materials can incorporate a host of applicable microorganisms that can be preserved, stored, and deployed in any number of industrial, agricultural, municipal, consumer, or research applications. As such, disclosed technology can provide an approach for adding microbial consortia to a wide range of environments including bodies of water and soil, as well as to urban environments. This opens a wide range of markets and makes the end users governmental affiliates, commercial businesses, and potentially even individuals. Applications include those in the oil, gas, and mining industry, agricultural applications, wastewater treatment systems, defense contractors, biotechnology, and bioremediation projects where effective delivery of active microbial species is critical.

The present invention may be better understood with reference to the examples, set forth below.

### Example 1

Commercially available FGCs were provided by GlassWRX, Beaufort, SC. To ascertain the basic properties of the material, it was analyzed via powder x-ray diffraction (PANalytical X'Pert³). The scan was performed from 2 to 55 degrees 2⊖, with a scan speed of 1 second and 0.2° step size. The surface area was analyzed via physisorption (Anton Parr autosorb™ iQ) using krypton gas and the Brunauer-Emmett-Teller (BET) model. Micrographs were collected to observe surface characteristics of the material with a scanning electron microscope (Zeiss Supra™ 40 VP, 5.1 mm working distance, 1.0 kV, high vacuum).

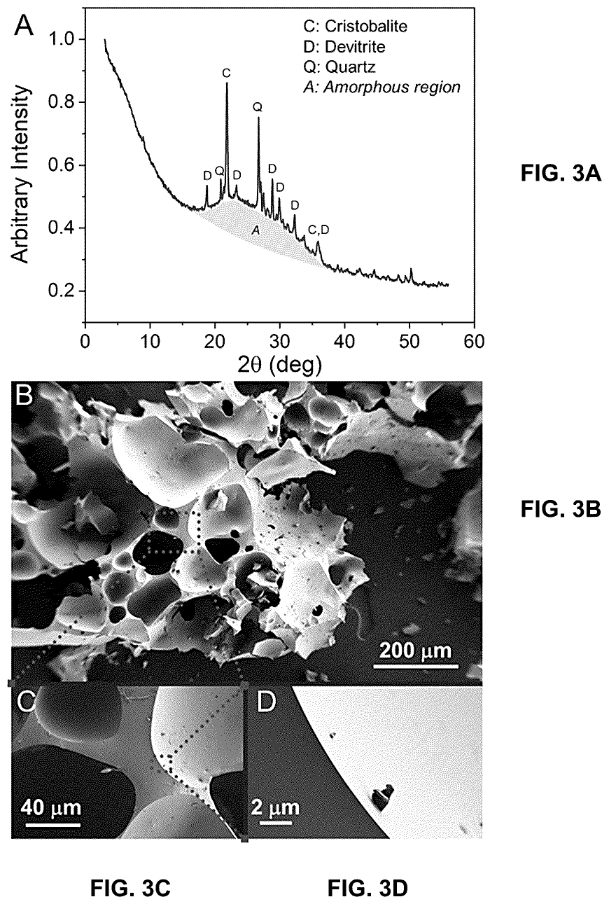

The FGCs were observed to be largely amorphous (>65%) with inclusions of cristobalite (a high temperature silica polymorph (SiO₂)) quartz (SiO₂), and devitrite (an orthorhombic silicate (Na₂Ca₃Si₆O₁₆)) crystallized during the cooling of the soda-lime silicate-based FGC after synthesis. The x-ray diffraction pattern for the material is shown in FIG. 3A. The peaks highlighted in FIG. 3A correspond to the peaks of cristobalite, devitrite, and quartz with the characteristic amorphous hump ranging from 2e which is common to all amorphous glass products. An amorphous phase was also observed (centered on 2θ˜28°, FWHM˜10°), which is indicative of the glass from which the FGC is synthesized. The chemical composition of the FGCs was assumed to be equivalent to commercial soda-lime silicate, typical composition of this glass is 70-wt. % SiO₂, 12-16 wt. % of Na₂O, 10-15 wt. % CaO, and a balance of multiple other oxides such as Al₂O₃, FeO, Fe₂O₃, K₂O, MgO, and/or other trace amounts of additional oxides. The surface area of the FGC material was measured to be 0.3 m²/g.

Corresponding SEM micrographs, FIG. 3B, FIG. 3C, and FIG. 3D, show the texture and high porosity of the FGC. Smooth edges still show texturing and pitting, which is ideal for microbial attachment. As can be seen in the micrographs, the surfaces are smooth, and the pore sizes range from approximately 100-500 μm. This FGC pore size is ideal for colonization of microorganisms that range in size from 1-2 μm.

BioTiger™ bacterial consortium, Bacillus thuringiensis (ATCC 35646) and Escherichia coli k12 (ATCC 25404), was routinely cultured on Reasoner's 2A (R2A; Fisher Scientific) media containing the following per liter of water: 0.5 g casein acid hydrolysate, 0.5 g dextrose, 0.3 g K₂HPO₄, 0.024 g MgSO₄, 0.5 g proteose peptone, 0.3 g sodium pyruvate, 0.5 g soluble starch, and 0.5 g yeast extract. The media was buffered to a pH of 7.2±0.2 at 25° C. Routine bacteria stocks were cultured at room temperature on a shaker plate at 100 rpm. All additional experiments using bacteria were conducted using R2A media or agar prepared with 15 g/L agar (Fisher Scientific), as necessary. To test for compatibility with different cell types an environmental algal sample was used. The algal sample was maintained using Bushnell Haas broth containing the following per liter of water: 0.2 g MgSO₄, 0.02 g CaCl₂), 1.0 g KH₂PO₄, 1.0 g K₂HPO₄, 1.0 g NH₄NO₃, and 0.05 g FeCl₃ adjusted to a final pH of 7.

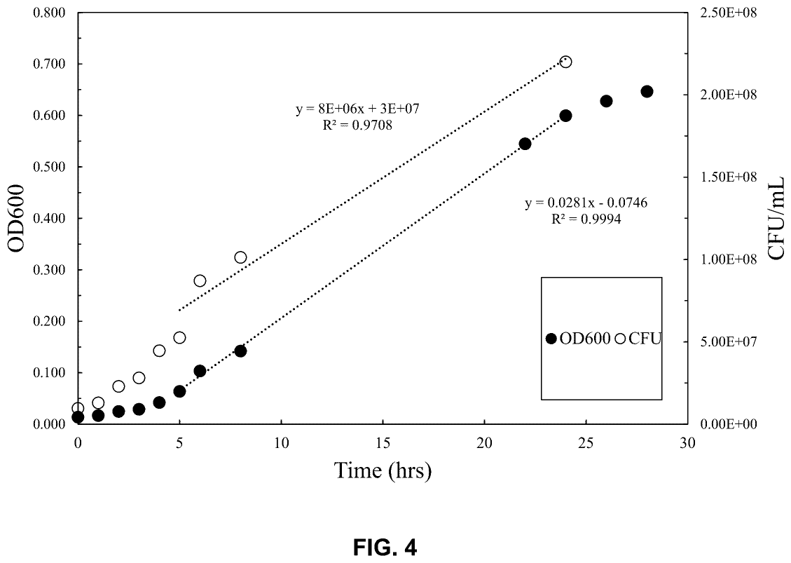

To facilitate the BioTiger™ growth measurement, fresh cultures were inoculated and monitored at 0, 1, 2, 3, 4, 5, 6, 8, 22, 24, 26, 28, and 48 hours. At each of these time points, the optical density at 600 nm (OD600) was measured using a GENYSIS™ Vis20 (Thermo Scientific™) spectrophotometer. Concurrently, samples were serially diluted, and 100 μL of the solution was plated on R2A agar in triplicate and incubated for three days. All samples were grown in R2A at room temperature on a rotary shaker plate at 100 rpm. Dilutions outside of the optimal range of colony forming units (CFUs), 20-200 were discarded while the optimal plates were counted to correlate the optical density at 600 nm with CFUs. The resulting growth curves are illustrated in FIG. 4. As indicated, trend lines calculated from 5-24 hours show a linear growth with an R² of 0.9994 and 0.9708 for the optical density and CFU samples, respectively. Error bars were within the size of the symbols shown.

To determine the effectiveness of FGCs as a substrate for biofilm growth, 3 g of sterile, pebble sized (φ of −3 to −4 (approximately 1 cm in diameter)) FGCs were placed into 100 mL of sterile R2A media. This media was then inoculated with 1 mL of log phase cells. The samples were incubated at room temperature on a rotary shaker plate at 100 rpm for one week. After samples had become laden with cell mass, a sterile solution of 80% glycerol was added bringing the overall concentration of glycerol to 20% following which the samples were allowed to rest for five minutes. The FGCs were then removed from the solution and placed in a −80° C. freezer (Thermo Scientific™). After freezing, the samples were lyophilized (Labconco™ Benchtop 2.5 L freeze dryer) and freeze-dried over a 48-hour period. Samples were then stored in a refrigerator at 4° C. Samples were examined using an Olympus SZX16 stereoscope (Evident™, Tokyo) to determine whether biofilm growth was a success.

To determine the success of storage, 10 of the pebble sized treated FGCs were placed into 100 mL of R2A media 1, 3, 7, 14, 21, 28, 56, and 84 days after lyophilization. Growth was indicated by an increase in turbidity, OD600 over the course of 72 hours at room temperature and shaking at 100 RPM.

A list of tested microorganisms is provided in Table 2, below. The table includes the name, American type culture collection (ATCC) number, and cell type along with a positive or negative indicator for growth in conjunction with the FGCs, whether the sample was preserved, and if it positively regrew.

TABLE 2

| | | Cell | | Pre- | Re- |
|---|---|---|---|---|---|
| Name | ATCC | Type | Growth | served | grown |
| Escherichia coli K12 | 47076 | Gram− | + | + | + |
| BioTiger ™* | N/A | Consortia | + | + | + |
| Bacillus thuringiensis | 33679 | Gram+ | + | + | + |
| Chlorella sp | N/A | Eukaryotic | + | + | + |
| Lecanicillium sp | N/A | Eukaryotic | + | + | + |
| Sphingomonas sp BPH | PTA- | Gram− | + | + | + |
| | 5574 | | | | |
| Bacillus cereus | 13061 | Gram+ | + | + | + |
| Shewanella oneidensis | 700550 | Gram− | + | + | + |
| Pseudomonas putida | 47054 | Gram− | + | + | + |

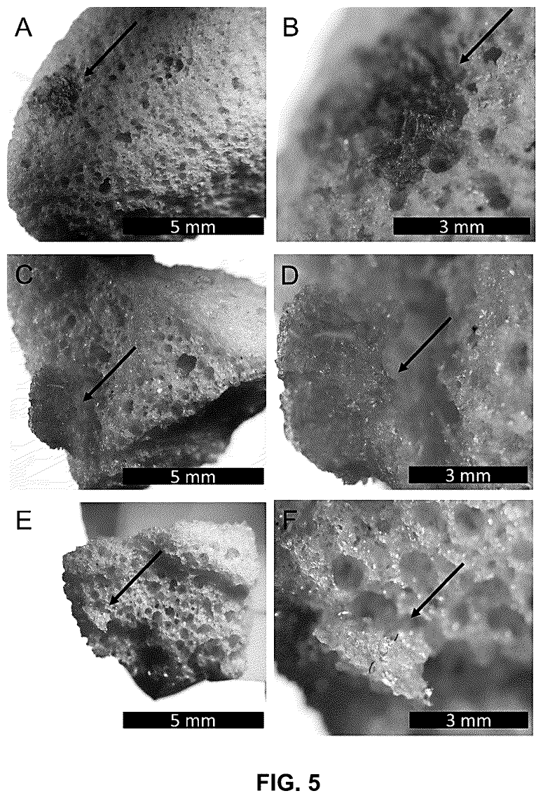

Optical analyses were carried out using three of the test microorganisms, the BioTiger™ consortia, Bacillus thuringiensis, and the algae. Preserved biofilm formations can be seen in FIG. 5. The biofilms have been highlighted in the images with a black arrow. The biofilms range from 1-3 mm in radius and were stable under the stereoscope. The algal cells (A and B), BioTiger™ cells (C and D) and Bacillus thuringiensis cells (E and F) are seen on the surface of the FGCs, attached to the porous structure of the FGC.

While none of the tested microorganisms were negatively impacted by the presence of the FGCs, it was desirous to determine whether the preservation process left the cells viable. Each of the tested microorganisms were then regrown, checking for turbidity as a positive confirmation of successful preservation.

Over approximately three months, lyophilized FGC samples stored at 4° C. were tested for success. FGCs preserved with the BioTiger™ consortium, Bacillus thuringiensis, and the algae were tested for growth after 1, 3, 7, 14, 21, 28, 56, and 84 days of storage. Each sample tested positive for an increase in turbidity within the media within 24 hours for the bacterial samples and within one week for the algae.

To determine the rate of regrowth of preserved FGCs, a series of FGCs were incubated at 0, 2, 6, 24, 48, 72, and 196 hours with BioTiger™ prior to the preservation process, and then preserved as described above. Ten preserved FGCs from each incubation time were placed in 75 mL of sterile R2A broth and the OD600 was measured over time using a visible light spectrophotometer. The experiment was performed in duplicate.

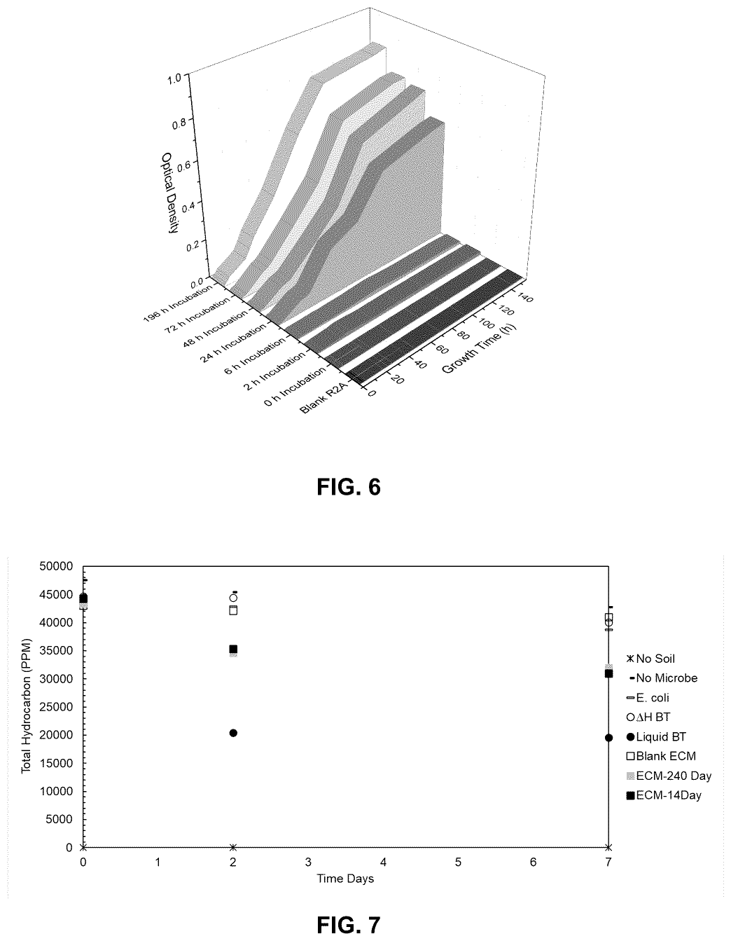

The rate of regrowth from the preserved state was tested by comparing incubation times prior to preservation at 0, 2, 6, 24, 48, 72, and 196 hours with BioTiger™. Growth occurred over several days in R2A media on a rotary shaker at 100 RPM. The growth was tracked using optical density at 600 nm. Different incubation times were tested in parallel to determine how long FGCs would need to be incubated with bacterial cultures prior to undergoing the preservation technique. The results (FIG. 6) show the average of duplicate experiments. As indicated, there were two distinct trends amongst the samples. Samples which were incubated for 0, 2, and 6 hours show no, or almost no, bacterial regrowth over the incubation period of nearly a week. Samples which were incubated for 24, 48, 72, and 196 hours show a linear growth trend between approximately 6 and 96 hours, with an initial lag phase, which then plateaus between 100 and 140 hours. Unsurprisingly, samples that were incubated longer prior to preservation resulted in the fastest reestablishment of a microbial community.

### Example 2

Hydrocarbon contaminated soil was procured from a private environmental firm operating in Ecuador. The soils were lyophilized prior to shipment and stored in a 4° C. refrigerator prior to use. The size profile was estimated by washing the soils in acetone and DI water to remove the hydrocarbons and then sieved with a 200-mesh sieve, corresponding to a particle retention size of 0.075 mm. Soil texture was also qualitatively determined using the texture-by-feel method. Soil pH was determined via EPA Method 9045D using a Thermo Scientific™ Orion Star™ A121 pH meter, calibrated with commercially available standards of 4.01, 7.00, and 10.01. The average soil pH, as determined by the US EPA method, of three replicates was 7.09 with less than 1% difference between measurements.

The native bacterial population of the soil was estimated by counting the CFU. A soil slurry was prepared by adding 1 g of soil to 100 mL of water. R2A agar plates were prepared at the following dilutions for plate counting: no dilution, 10⁻¹, 10⁻², 10⁻³, 10⁻⁴, 10⁻⁵, 10⁻⁶, and 10⁻⁷. 100 mL of each dilution and a sterile water control were spread and allowed to incubate at room temperature for 48 hr. and counted. The estimated community, an average of triplicates, was 4.2×10⁵ cells/g microorganisms.

The soil texture was analyzed for sand content and qualitatively assessed. The average of duplicate sand size analyses resulted in a recovery of 68.8% sand by weight. When examined by hand, the texture held together in a wetted ball and formed a small ribbon, suggesting that the texture was a comparatively high clay sandy loam, which corresponds to the measured sand content.

All microbial media components were purchased from Difco (Detroit, MI). BioTiger™ and constituent cultures were maintained using Reasoner's 2A media. Escherichia coli K₁₂ (ATCC 10798) was grown on Luria-Bertani liquid media to serve as a negative control. BioTiger™-laced FGCs were prepared as described in Example 1. Following inoculation, microbial growth was allowed for seven days, followed by preservation as described above and stored at 4° C. for either 14 days or 240 days.

5 g of soil (small stones and sticks were removed) was amended with 10 mL of sterile Bushnell Haas media containing each of the following per liter: 0.2 g MgSO₄, g CaCl₂), 1.0 g KH₂PO₄, 1.0 g (NH₄)₂HPO₄, 1.0 g KNO₃, and 0.05 g FeCl₃, and further enriched with 0.2% yeast extract to stimulate bioremediation. Fresh BioTiger™ cells were washed by centrifuging (Eppendorf; Hamburg, Germany) at 5000× g three times with sterile Bushnell Haas media. One portion was heat killed to serve as a negative control. One mL of approximately 1.0×10⁸ cells, as determined by OD600 (Thermo Scientific™, Waltham, MA) was inoculated into the positive control sample, while the same was done with the negative control of Escherichia coli, and the heat killed cells. For each of the soil slurries treated with FGCs, 5 of the untreated, 14, and 240-day FGC samples were placed into the slurry. Resulting materials were incubated at room temperature on a rotary incubator at 100 rpm. Samples were taken at 0, 24, and 48 hrs.

Samples were analyzed for total hydrocarbon using a PetroFLAG® analyzer for total hydrocarbon analysis. The analysis was performed as described in the kit. In short, 0.5 g of soil sample was placed into a 50 mL polypropylene tube, and a vial of the provided solvent was added, mixed, and rested for 5 minutes. This solution was then decanted into a syringe and filtered (0.22 micron) into the development solution. This solution rested for 10 minutes, became turbid in the presence of the hydrocarbons, and was measured using the analyzer. Blanks and standards were run as provided prior to each sample analysis.

Degradation of crude oil hydrocarbons were carried using BioTiger™ as a liquid culture as well as a preserved biofilm on the surface of FGCs stored over the two different time periods. With the exception of the no contaminated soil blank, which unsurprisingly showed no present hydrocarbons, each of the controls showed some loss of hydrocarbons. Reduction of overall hydrocarbons for the control samples were as follows:
Soil without the addition of any microorganism-10.1%,
Sample inoculated with E. coli K₁₂-10.9%
Heat killed BioTiger™ sample-10.3%
FGCs without preserved biomass-4.9%

Results are also shown in FIG. 7. Each sample result is the average of duplicate experiments. On FIG. 7, the term “No Soil” refers to a sample containing only media, “No Microbe” indicates no inoculation though the soil contained its native community, “E. coli,” “Blank FGC,” “FGC-240 Day,” and “FGC-14 Day, are as described above. “Liquid BT” refers to BioTiger™ utilized as a liquid preparation, with no solid substrate carrier, and “ΔH BT” is the heat killed BioTiger™ sample. Error bars were smaller than symbols shown.

The results of the control samples are likely due to abiotic physiochemical processes, mostly evaporation of many of the lighter aromatics that will volatilize well below room temperature, which was the incubation temperature of the samples.

The three experiment sample sets each showed significantly higher loss of total hydrocarbons. The liquid BT showed the highest overall decrease in total hydrocarbons over the course of seven days reaching a total decrease of 55.9%. The 14 and 240 BioTiger™-FGCs showed a total decease in hydrocarbons by a total of 26.2% and 30.0%, respectively (FIG. 7). Although the FGC-preserved BioTiger™ was not as effective as the liquid culture, it should be noted that a larger amount of live biomass was added via the liquid culture. The liquid BT culture was added directly from a freshly grown culture, while the preserved cells are difficult to enumerate for several reasons including: difficulty in determining live cells vs dead cells and the presence of preserved extra polymeric substances, which adds weight but not live cells.

FIG. 7 also makes clear that much of the hydrocarbon loss in the liquid samples occurred within the first two days, while the samples which were preserved did not reduce the concentration as much in those first 48 hours. This apparent delay was likely due to the difference in metabolism of fresh cultures versus samples which had been preserved. Notably, there was little difference between the two sets of FGCs. One was prepared fresh and was stored in a refrigerator for 14 days prior to use—the older, however, had been prepared months beforehand and was stored in a refrigerator until the initiation of these experiments. These results demonstrate that not only do the BioTiger™ cultures survive the process but that they were only minimally affected by storage over a period of several months.

While certain embodiments of the disclosed subject matter have been described using specific terms, such description is for illustrative purposes only, and it is to be understood that changes and variations may be made without departing from the spirit or scope of the subject matter.

What is claimed is:

1. A microorganism storage and delivery system, comprising:
a solid, porous substrate comprising a glass-based material; and
a lyophilized microorganism adhered at a surface of the solid porous substrate.

2. The system of claim 1, wherein the solid, porous substrate comprises a foamed glass ceramic.

3. The system of claim 1, wherein the solid, porous substrate comprises a natural or synthetic foamed glass.

4. The system of claim 1, wherein the lyophilized microorganism is a component of a lyophilized biofilm.

5. The system of claim 1, wherein the microorganism comprises a bacterium.

6. The system of claim 5, wherein the microorganism comprises one or more oil degrading bacteria.

7. The system of claim 1, wherein the microorganism comprises an alga or a fungus.

8. The system of claim 1, wherein the solid, porous substrate is in the form of an aggregate including a plurality of individual porous pieces.

9. The system of claim 1, wherein the system comprises a microbial consortium that includes the lyophilized microorganism.

10. A method for processing a microorganism for delivery to a site of use, the method comprising:
culturing the microorganism in the presence of a solid, porous substrate comprising a glass-based material, wherein upon the culturing, the microorganism is adhered to the substrate;
lyophilizing the adhered microorganism;
storying and/or transporting the solid, porous substrate and the adhered lyophilized microorganism to a site; and
deploying the solid, porous substrate at the site, wherein following the deployment, the lyophilized microorganism grows and develops.

11. The method of claim 10, wherein the microorganism is adhered to the substrate in a biofilm.

12. The method of claim 10, further comprising culturing a microbial consortia in the presence of the solid, porous substrate, the microorganism being a component of the consortia.

13. The method of claim 10, wherein the solid, porous substrate and the adhered lyophilized microorganism is stored and/or transported at standard temperature and pressure conditions.

14. The method of claim 10, wherein the solid, porous substrate and the adhered lyophilized microorganism are deployed in conjunction with a microorganism nutrient.

15. The method of claim 14, wherein the solid, porous substrate comprises the microorganism nutrient.

16. The method of claim 10, further comprising collecting the solid, porous substrate at the site.

17. The method of claim 10, wherein the site comprises a contaminated soil.

18. The method of claim 10, wherein the site comprises a contaminated water.

19. The method of claim 10, further comprising modifying a characteristic of the solid, porous substrate and/or the adhered microorganism following the deployment.

20. The method of claim 19, wherein the modified characteristic comprises the buoyancy of the solid, porous substrate.
